(19) United States
(12) Patent Application Publication
(10) Pub. No.: US 2022/0096700 A1
(43) Pub. Date: Mar. 31, 2022

(54) DEVICE THAT USES ULTRAVIOLET LIGHT TO PURIFY AIR

(71) Applicant: Apache Industrial Services, Inc., Houston, TX (US)

(72) Inventor: David Bretherton, Humble, TX (US)

(21) Appl. No.: 17/112,324

(22) Filed: Dec. 4, 2020

Related U.S. Application Data
(60) Provisional application No. 63/084,129, filed on Sep. 28, 2020.

(51) Int. Cl. A61L 9/20

(52) U.S. Cl. CPC A61L 9/20; A61L 2209/14; A61L 2209/12

(57) ABSTRACT

Implementations of the present subject matter are directed toward a device for purifying air. The device for purifying air may include a conduit, a sheet, a light emitting diode and a fan. The conduit may have an inlet, an outlet and a plenum that couples the inlet to the outlet. The sheet may be at the inlet within the conduit and may be configured to filter bio aerosols from air or other fluid entering the conduit. The light emitting diode may be within the conduit and the fan may be positioned at the outlet.

### CROSS-REFERENCE TO RELATED APPLICATIONS

This application claims the benefit of priority under 35 U.S.C. § 119(e) to U.S. Provisional Patent Application No. 63/084,129, filed on Sep. 28, 2020, the contents of which are hereby incorporated in their entirety.

### FIELD

The present disclosure is directed toward a breathing apparatus that uses ultraviolet light emitting diodes to reduce health risk from airborne pathogens.

### BACKGROUND

This section is intended to provide background information to facilitate a better understanding of various technologies described herein. As the section's title implies, this is a discussion of related art. That such art is related in no way implies that it is prior art. The related art may or may not be prior art. It should therefore be understood that the statements in this section are to be read in this light, and not as admissions of prior art.

People are frequently at risk of being infected by airborne pathogens. For the general population, common pathogens include coronavirus, common cold viruses (e.g., rhinoviruses) and influenza virus. Sub-groups of the population, such as hospital patients and hospital employees, may be exposed to, and therefore be at risk of infection by, other pathogens for example Methicillin Resistant Staphylococcus aureus (MRSA) or Mycobacterium tuberculosis (TB). Hospitalized patients are particularly likely to have some level of immuno-suppression, such as elderly people, people with congenital immunodeficiency, people undergoing or shortly following cancer treatment or organ transplantation, and therefore be susceptible to infection. Current methods of preventing infections include vaccination of at-risk groups and/or the wearing of personal protective equipment (PPE) such as face masks, which include air filters.

Acute respiratory infection (ARI) causes millions of deaths every year. In the event of an ARI pandemic or other emerging respiratory disease such as severe acute respiratory syndrome (SARS), measures are preferably taken immediately to reduce the infection rate, rather than wait for a targeted vaccine or antiviral drug to be developed. Wearing a facemask is a widely accepted, non-pharmaceutical method to reduce the risk of respiratory infection.

Examples of common facemasks include disposable surgical facemasks and N95 respirators. This type of facemask reduces transmission of airborne pathogens by preventing a person from directly touching his nose and mouth with dirty hands and by containing large liquid droplets expelled during sneezing or coughing. This type of facemask is unable to disinfect the air being inhaled or exhaled, and typically cannot block airborne viruses, most of which are smaller than 0.3 microns and can pass through the pores in the fabrics of this type of facemask. In addition, because the main air passageway of the facemask is blocked by one or more layers of fabric, this type of facemask is generally uncomfortable to wear, which may discourage people from using facemasks. Furthermore, if the mask is not face-fitted, a significant amount of air can leak through the periphery of the mask, significantly reducing the mask's effectiveness and leading to other inconveniences such as fogging of lenses in cold weather for eye-glass wearers from leakage of moist air.

Current personal protective equipment includes face masks such as surgical masks and P3 face masks. Such masks include air filters. Examples of face masks include the FFP3 cone style face mask with valve. Such a mask typically has a polymer, which is lightweight and offers good breathability. The polymer may be ethylene propylene diene monomer (EPDM) rubber. The mask also typically has a melt blown filter media, which has a bacterial filter efficiency of 99% for bacteria of 3 μm size. This provides good protection from air-borne bacteria. The mask also typically has a particle filtration efficiency of 99% for 0.3 μm particles. The mask also typically includes a compliant cuff of a soft, latex-free material, which provides a seal between the mask and the face of the user with good skin comfort. These masks provide a physical barrier between the user and the air and therefore prevent bacteria from being breathed in by the user. The masks also tend to become blocked, which causes breathing difficulties and can result in the mask leaking thus allowing bacteria to enter the user.

Surgical masks protect the wearer from inhaling microorganisms and body fluids, which may be exhaled by nearby persons. A problem of such masks is that they have a high resistance against air flow and can therefore make breathing difficult. Furthermore, if not fitted carefully to the face of the user, such masks leak and therefore do not provide the protection expected and required. They also provide limited or no protection against pathogens transmitted as aerosols, that is particles with sizes of less than or equal to 5 μm diameter.

Current devices work by preventing microorganisms from contacting and/or being inhaled by the user of the device. That is, the devices are configured to prevent microorganisms from exiting the device. Such prevention may be achieved through the inclusion of a barrier such as a filter. A disadvantage of use of a filter is that it may become clogged and therefore prevent air from passing through the filter and or result in leakage around the filter thus allowing microorganisms to pass around the filter and consequently reach the user and thus rendering the device useless.

Vaccinations are possible, however, there are drawbacks associated with vaccinating at-risk groups against infection. For example, it is not always possible to know which pathogens are likely to be present. Also, it can be difficult to predict the general type and precise strain of pathogen which is likely to occur. Furthermore, it can be difficult to ensure that all at-risk people are vaccinated. Also, there might not be a vaccine available for the infectious organism in question; and a vaccine may not be suitable for certain groups of people such as immuno-suppressed individuals.

### SUMMARY

Some implementations of the present subject matter are directed toward a device for purifying air. The device for purifying air may include a conduit, a sheet, a light emitting diode (“LED”) and a fan. The conduit may have an air inlet, an air outlet and a plenum that couples the air inlet to the air outlet. The sheet may be at the air inlet within the conduit and be configured to filter bio aerosols from air entering the conduit. The light emitting diode may be within the conduit and the fan may be positioned at the air outlet.

Implementations of the device for purifying air may further include a second plenum in the conduit that is coupled to the air inlet. At least one of the plenum and the second plenum may be a substantially flexible tube. A second air outlet may be at an end region of the second plenum. The end region of the second plenum may oppose the end region of the plenum having the air inlet. A second fan may be at the second air outlet.

Implementations of the device for purifying air may further include a substrate that is substantially surrounded by the conduit. The substrate may include the LED on at least one planar surface of the substrate. The substrate may include a plurality of LED's on a first surface of the substrate and a second plurality of LED's on second surface of the substrate. The second surface may be the surface of the substrate that opposes the first surface of the substrate.

The air inlet may provide air flow through the air inlet in a direction substantially perpendicular to air flow through an inlet of the plenum and/or the second plenum. A second air inlet may be positioned on the device to provide air flow through the second air inlet in a direction substantially perpendicular to air flow through an inlet of the plenum and/or second plenum.

Further implementations of the present subject matter may be directed toward a garment for purifying air. The garment for purifying air may include a fabric enclosure, and a device for purifying air. The device for purifying air may be substantially enclosed by the fabric enclosure. The device for purifying air may include a conduit, a sheet, a light emitting diode and a fan. The conduit may have an air inlet, an air outlet and a plenum that couples the air inlet to the air outlet. The sheet may be at the air inlet within the conduit and be configured to filter bio aerosols from air entering the conduit. The light emitting diode may be within the conduit and the fan may be positioned at the air outlet.

The present subject matter may be implemented in a method of purifying air. The method of purifying air may include filtering air entering an inlet of a container through a filtering sheet at the inlet of the container and providing a dose of energy, i.e., at least 600 μJ/cm² of energy, to impede reproductive capability of microorganisms (microbes, bacteria, viruses, mold, etc.) in the air in the container throughout the container. The method of purifying air may further include controlling a fan at an outlet of the container to pull air through a conduit and expelling air from the container by using a fan positioned at an outlet of the container. The dose of energy may be provided by at least one light emitting diode or a plurality of light emitting diodes.

The above referenced summary section is provided to introduce a selection of concepts in a simplified form that are further described below in the detailed description section. Additional concepts and various other implementations are also described in the detailed description. The summary is not intended to identify key features or essential features of the claimed subject matter, nor is it intended to be used to limit the scope of the claimed subject matter, nor is it intended to limit the number of inventions described herein. Furthermore, the claimed subject matter is not limited to implementations that solve any or all disadvantages noted in any part of this disclosure.

### BRIEF DESCRIPTION OF THE DRAWINGS

Implementations of various techniques will hereafter be described with reference to the accompanying drawings. It should be understood, however, that the accompanying drawings illustrate only the various implementations described herein and are not meant to limit the scope of various techniques described herein.

FIG. 1A illustrates a first perspective of an implementation of a garment for purifying air;

FIG. 1B illustrates a second perspective of the implementation of the garment for purifying air illustrated in FIG. 1A;

FIG. 2 illustrates an implementation of a device for purifying air;

FIG. 3A illustrates a perspective of a conduit of the device for purifying air;

FIG. 3B illustrates a cross-sectional view of the conduit of the device for purifying air illustrated in FIG. 3A;

FIG. 3C illustrates an exploded view of the conduit illustrated in FIG. 3A;

FIG. 4 illustrates an end region of the conduit illustrated in FIG. 3A;

FIG. 5A illustrates a tube of the conduit of the device for purifying air;

FIG. 5B illustrates an LED strip within an inlet of the conduit of the device for purifying air;

FIG. 5C illustrates the inlet of the conduit of the device for purifying air of FIG. 5A and the LED strip of FIG. 5B; and

FIG. 6 illustrates an electrical connector of the garment illustrated in FIGS. 1A and 1B.

### DETAILED DESCRIPTION

Implementations of the present subject matter may be provided to eliminate bioaerosols from ambient air surrounding a person thereby providing clean air to the person. Surfaces of harmless airborne particles often host living organisms such as bacteria, mold and viruses. These particles are known as bioaerosols.

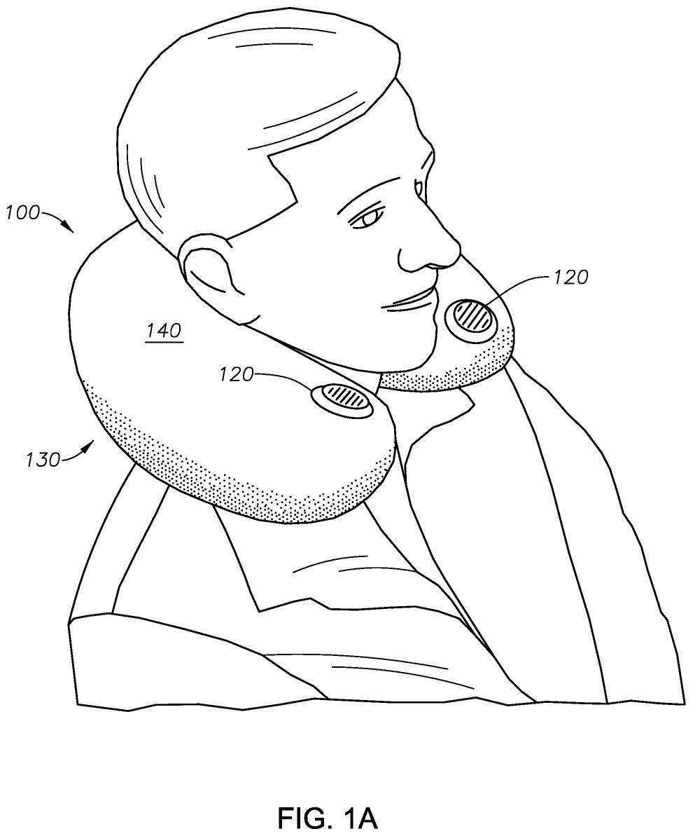

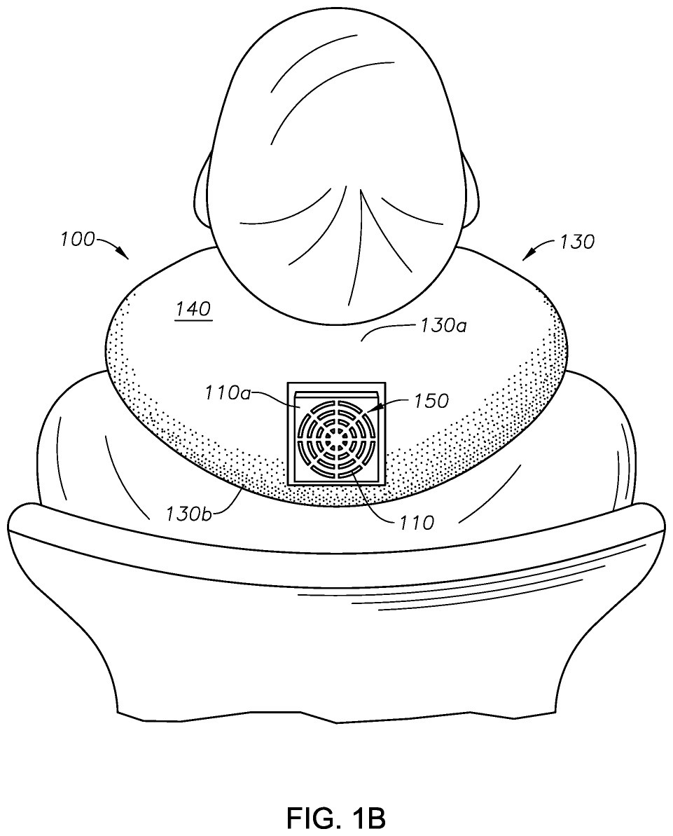

FIGS. 1A-1B illustrate an implementation of an air purifier 100. The air purifier 100 may include at least one air inlet 110, at least one air outlet 120 and a cushion 130.

The cushion 130 may include a flexible outer surface 140 that substantially surrounds an inner material (not shown) that may be soft, e.g., compressible, elastic, resilient, soft, spongy, etc. For example, except for at least the air inlet 110 and the air outlet 120, the flexible outer surface 140 may completely surround the inner material.

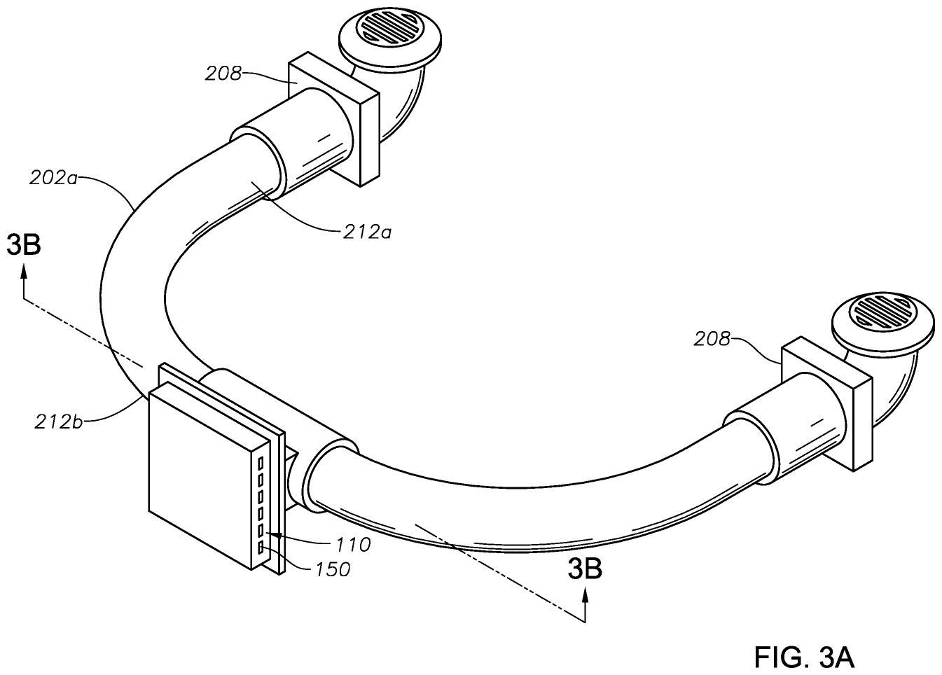

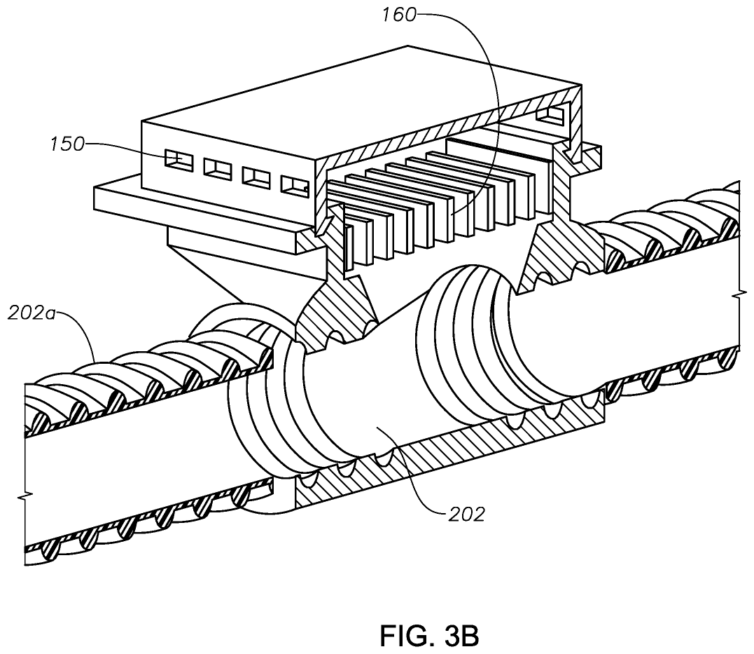

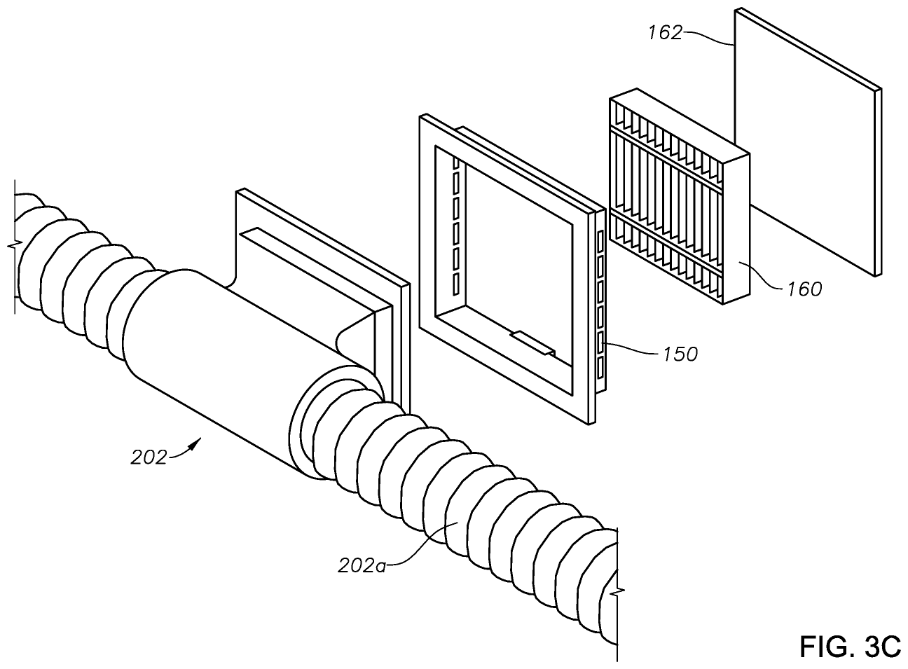

The air inlet 110 may include an air inlet housing 110a, a vented opening 150 and an air filter 160 (shown in FIG. 3B). For example, the air filter 160 may be directly or indirectly coupled to or connected to the vented opening 150 to clean air entering the air purifier 100 through the air inlet 110. The air filter 160 may be a high efficiency particulate air (HEPA) filter that has a minimum efficiency reporting value (MERV) rating of at least 20, i.e., the air filter 160 is configured to remove about ninety-nine point nine percent (99.9%) of all bio aerosols having a diameter of about 0.3 microns or less and about ninety percent (90%) of all bio aerosols having a diameter of about 0.2 microns or less.

In some implementations, the vented opening 150 may be on an intermediate portion of the rear of the air purifier 100 so that air at a posterior area of a person wearing the air purifier 100 is pulled into the air inlet 110. In other implementations, the air inlet 110 and the vented opening 150 may be on a top surface 130a or bottom surface 130b of the air purifier 100 so that air immediately above or below the wearer's neck, respectively, is pulled into the air inlet 110. In an implementation having the vented opening 150 at the top surface 130a or the bottom surface 130b of the air purifier 100, the air inlet may operate like a snorkel, pulling in air from above or below the air purifier 100. In yet further implementations, multiple vented openings may be on any combination of the rear of the air purifier 100, the top surface 130a of the air purifier 100 and/or the bottom surface 130b of the air purifier 100. Respective air filters may be associated with each vented opening of the multiple vented openings.

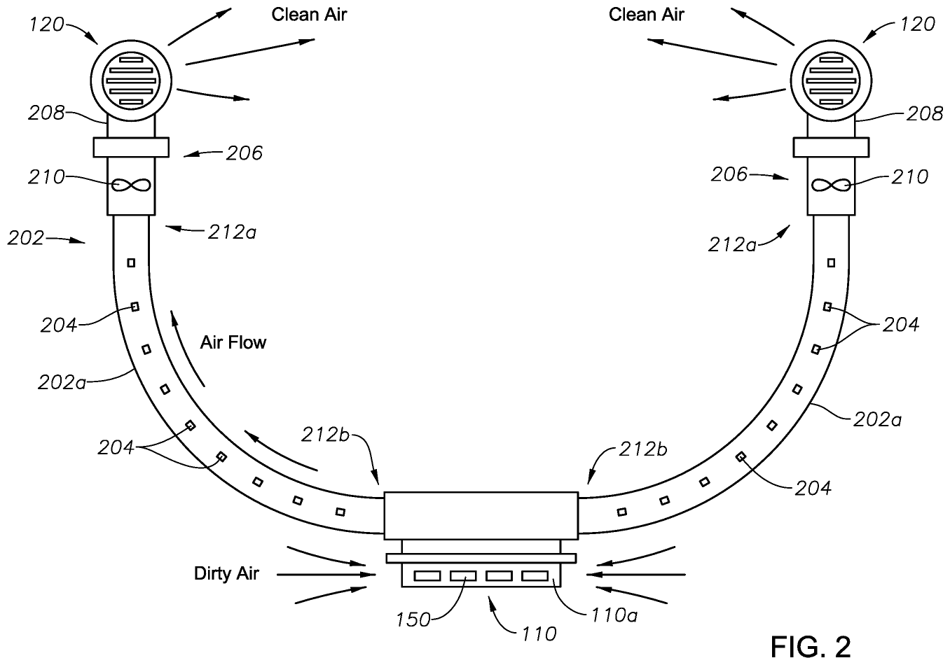

FIGS. 2, 3A-3C and 4 illustrate a conduit 202. The interior of the air purifier 100 may encapsulate at least one conduit 202. The conduit 202 may originate at the air inlet 110. The conduit 202 may terminate at the outlet 120. The conduit 202 may include a tube 202a and a plurality of ultraviolet LED lights 204.

A terminal region 206 of the conduit 202 may include the air outlet 120, a terminal region housing 208, a fan 210, a first terminal end 212a of the tube 202a and a second terminal end 212b of the tube 202a. The fan 210 may be located at any point in the conduit 202, in the terminal region housing 208 or in the tube 202a. If the fan 210 is located in the conduit 202, the fan 210 may be at or near the first terminal end 212a of the tube 202a.

The terminal region housing 208 may be attached to the first terminal end 212a of the tube 202a in any way that substantially creates an airtight seal with the tube 202a. For example, in some implementations, the tube 202a and the terminal region housing 208 may include threads that interact with each other to create a seal with threads on the first terminal end 212a of the tube 202a. In other implementations, the tube 202a and the terminal region housing 208 may be in an interference fit with each other. In yet other implementations, a gasket, O-ring or other type of sealing means may be used between the tube 202a and the terminal region housing 208.

The tube 202a may have any type of cross-sectional configuration. For example, the tube 202a may have a circular cross-section, a rectangular cross-section, a polygonal cross-section and/or any combination of regular and irregular cross-sections. The tube 202a may be a plenum, i.e., a chamber, that is sized to house air received from the inlet 110 for an amount of time sufficient to purify air in the conduit before it reaches the outlet 120. The tube 202a may be flexible or rigid. A rigid object, e.g., a rigid tube, may be an object that returns to its original form after deformation through application of force whereas a flexible object may be repositioned into a different form and remain in the different form after an applied force is removed from the object.

As illustrated in FIG. 2, the terminal region 206 of each conduit 202 may include the air outlet 120 and the fan 210. The air outlet 120 may be oriented in the terminal end region 206 such that air flow through the air outlet 120 is emitted toward the wearer's nose and/or mouth. Therefore, the conduit 202 may be long enough to extend from behind the wearer's neck to beyond and/or in front of the wearer's face.

The fans 210 in each of the terminal region housings 208, within the conduit 202, or within the tube 202a may be oriented so that the fans 210 pull air from the air inlet 110 through the conduit 202 and out toward the air outlet 120.

In some implementations, it may be possible to attach the terminal regions 206 of the conduits 202 to a face mask (not shown) that covers and hermetically seals to a person's face. For example, the air purifier 100 may have a gas mask type configuration that may be a full facial covering (covering the eyes, nose and mouth) or a partial facial covering (covering the nose and mouth).

The air filter 160 may be accessed and replaced by removing air filter cover 162. The air filter cover 162 may be secured to the air inlet housing 110a by a clip, a screw, a pin or any other fastening means. For example, once the air filter 160 reaches its maximum usage limit, a person may wish to replace the air filter 160 with a new air filter by unsecuring the fastening means and accessing the air filter and resecuring the air filter cover 162.

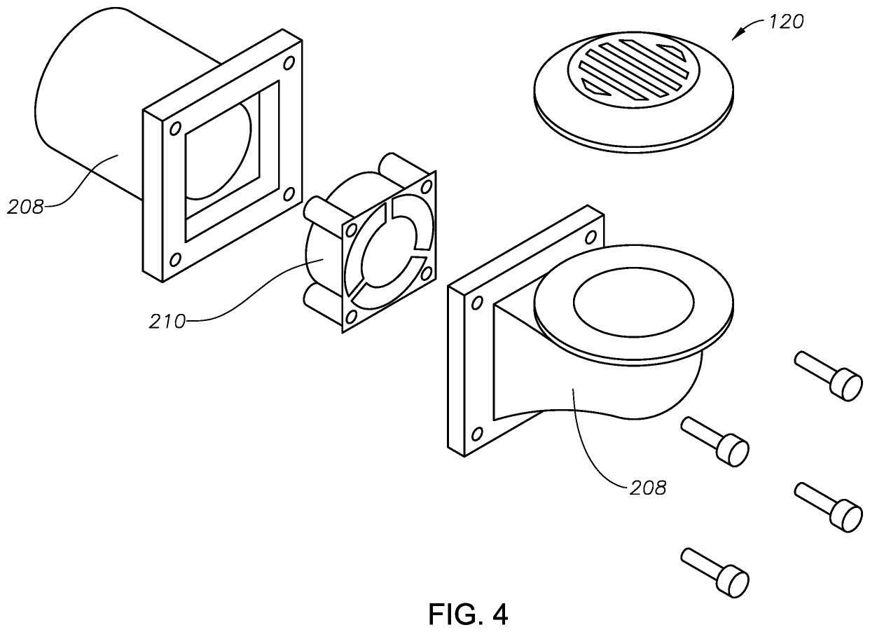

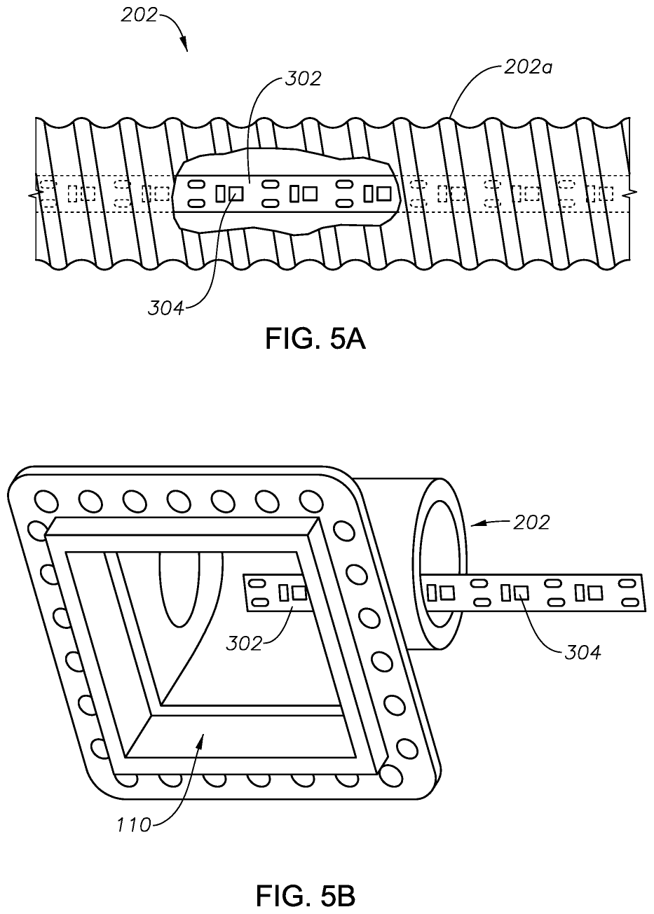

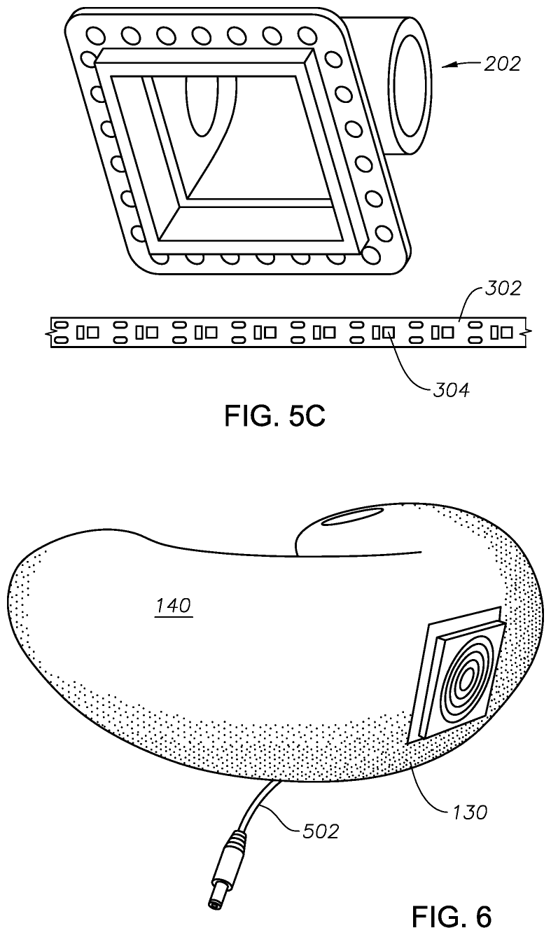

As illustrated in FIGS. 5A-5C, at least one flexible strip 302 may be included in the conduit 202 and tube 202a. The flexible strip 302 may include a plurality of light emitting diodes (LED's) 304. The flexible strip 302 may include opposing planar sides and both planar sides of the flexible strip may include LED's 304.

In some implementations, it may be possible to use a firm strip, e.g., an inflexible strip, in place of the flexible strip 302. In other implementations, it may be possible to use a flexible wire or cable or a firm wire or cable in place of the flexible or firm strip. In any of the implementations in which an alternative to the flexible strip is used, the LED's may be positioned on the flexible (or firm) strip (or wire/cable/etc.) in a manner that the light emitted from the LED's is capable of emanating around the flexible (or firm) strip (or wire/cable/etc.) as close to three hundred and sixty degrees as possible. For example, if a first LED is placed on a flexible strip in a manner that the flexible strip blocks emission of light, a second LED may be placed on the strip in a position that the second LED emits light in a direction that the first LED cannot emit light.

The LED's do not have to be placed on a strip, wire, cable, etc. They may instead be placed on an inner surface of the tube. For example, a first LED may be placed on a surface of the tube in a configuration that light is emitted toward a center of the tube. A second LED may be placed at a different position of the inner surface of the tube in a manner that light is emitted toward the first LED. To ensure that the LED's illuminate the entire interior of the conduit, the different position of the second LED may be a side of the tube that opposes the first LED or the different position of the second LED may be next to the first LED separated by a distance of between five and ten times the width of the LED.

A controller (not shown) may be included to control the fan speed, the intensity of each of the LED's, the number of illuminated LED's and the duration of illumination of each LED. For example, in a power save mode, it may be possible to control LED intensity and duration to conserve battery life. Further, one or both fans 210 may be intermittently activated to conserve battery life.

The LED's 304 and each of the fans 210 may be powered by an AC power source or by a DC power source. For example, a 1.5-volt battery pack may be included within the cushion 130 and made accessible therethrough for installation and replacement by the user.

As shown in FIG. 6, a cord 502 may lead to a controller that is external to the cushion 130. The controller may be connected to a touch pad (not shown), which may be in the flexible outer surface 140 or remote from the cushion 130 so that it can be handheld. The cord 502 may be a power cord for connecting to an AC power source to either recharge batteries within the cushion or to provide power directly to the fan(s) 210 and to the LED's 304. The cord 502 may also be attached to an external battery power source that may be integral with a handheld controller. A second and or a third cord (not shown) may be provided so that the cord 502 can be dedicated to one particular function, i.e., communication between LED's and a controller, and the second and/or third cord can be dedicated to a different function, i.e., power supply. Wi-fi and/or Bluetooth connections may be used in place of a cord.

In operations of implementations, dirty air (air containing microorganisms) entering the air purifier 100 through the inlet 110 may be first purified by the air filter 160 and may be further purified within the conduit 202. For example, as air (or other fluid such as a particular gas or liquid) entering the air purifier 100 passes through and then leaves the air filter 160, it passes along the conduit and passes over the plurality of LED's 304 that are emitting light in the UV-C radiation bandwidth. In some implementations, the air filter 160 and/or a second air filter (not shown) may be downstream of the plurality of LED's 304.

The UV-C light is used to deactivate living organisms in the air that can harm the wearer of the air purifier 100. For example, the amount of UV-C light (dosage of UV-C light) may be high enough to impede propagation of organisms in the air such as viruses, bacteria, mold and other microbes. This effect may be accomplished by damaging an organism's DNA, thereby deactivating its ability to reproduce.

The fans 210 may be used to draw the air past the filter 160, along the conduit 202. Clean air may then be blown by the fans into the user's breathing zone (e.g., a region of space proximate to the user's nose and/or mouth) to help reduce potential infection from viruses and bacteria in the environment, and reduce reactions from mold, mildew, pollen, etc.

Flow rate through the conduit may be controlled by the fans 210. The fans may be set at a speed to provide a UV-C light dosage to the air within the conduit 202 of at least about 600 μJ/cm². To provide enough of a dose of energy to deactivate any microorganisms, i.e., bacteria, mold, viruses and other microbes that enter the conduit 202, the tube 202a may have an inner diameter of about one inch. Any unwanted organisms may therefore be limited to a proximity of no more than about one-half inch from the LED's 304 to promote an environment that is hostile toward any virus that is within the conduit 202. It should be noted that a dose of energy that impedes propagation of unwanted organisms, e.g., deactivation of reproduction capability, may be about 600 μJ/cm² or it may be another higher or lower value depending on the targeted microorganisms. Specific quantities of UV-C energy that would be sufficient to be an impediment to propagation of particular mircoorganisms can be identified in existing publications.

The inner diameter of the tube 202a may be larger or smaller than one inch. However, the fan speed and/or number and/or intensity of LED's in the tube 202a should change to accommodate the changed inner diameter of the tube 202a. For example, a tube 202a having a larger diameter tube 202a, may require a slower fan speed or a higher number and/or intensity of LED's. Similarly, a tube 202a having a smaller diameter tube 202a, may allow for a faster fan speed and/or a smaller number and/or intensities of LED's.

The discussion above is directed to certain specific implementations. It is to be understood that the discussion above is only for the purpose of enabling a person with ordinary skill in the art to make and use any subject matter defined now or later by the patent “claims” found in any issued patent herein.

It is specifically intended that the claimed invention not be limited to the implementations and illustrations contained herein but include modified forms of those implementations including portions of the implementations and combinations of elements of different implementations as come within the scope of the following claims. It should be appreciated that in the development of any such actual implementation, as in any engineering or design project, numerous implementation-specific decisions may be made to achieve the developers' specific goals, such as compliance with system-related and business related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure. Nothing in this application is considered critical or essential to the claimed invention unless explicitly indicated as being “critical” or “essential.”

In the above detailed description, numerous specific details were set forth in order to provide a thorough understanding of the present disclosure. However, it will be apparent to one of ordinary skill in the art that the present disclosure may be practiced without these specific details. In other instances, well-known methods, procedures, components, circuits and networks have not been described in detail so as not to unnecessarily obscure aspects of the implementation.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the invention. The first object or step, and the second object or step, are both objects or steps, respectively, but they are not to be considered the same object or step.

The terminology used in the description of the present disclosure herein is for the purpose of describing particular implementations only and is not intended to be limiting of the present disclosure. As used in the description of the present disclosure and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context. As used herein, the terms “up” and “down”; “upper” and “lower”; “upwardly” and downwardly”; “below” and “above”; and other similar terms indicating relative positions above or below a given point or element may be used in connection with some implementations of various technologies described herein.

While the foregoing is directed to implementations of various techniques described herein, other and further implementations may be devised without departing from the basic scope thereof, which may be determined by the claims that follow. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

I claim:

1. A device for purifying air comprising:
a conduit having an air inlet, an air outlet and a plenum coupling the air inlet to the air outlet;
a sheet at the air inlet, the sheet configured to filter bio aerosols from air entering the conduit;
a light emitting diode (LED) within the conduit; and
a fan at the air outlet.

2. The device for purifying air as recited in claim 1, further comprising:
a second plenum in the conduit and coupled to the air inlet.

3. The device of claim 2, wherein at least one of the plenum and the second plenum is a tube.

4. The device for purifying air as recited in claim 2, further comprising:
a second air outlet at an end region of the second plenum opposing the air inlet; and
a second fan at the second air outlet.

5. The device for purifying air as recited in claim 1, further comprising:
a substrate substantially surrounded by the conduit, wherein the substrate includes the LED on at least one surface of the substrate.

6. The device for purifying air as recited in claim 5, wherein the substrate includes a plurality of LED's on each of opposing surfaces of the substrate.

7. The device for purifying air as recited in claim 1, wherein the air inlet is configured to provide air flow through the air inlet substantially perpendicular to air flow through an inlet of the plenum.

8. The device for purifying air as recited in claim 7, further comprising:
a second air inlet configured to provide air flow through the second air inlet substantially perpendicular to air flow through an inlet of the plenum.

9. A garment for purifying air comprising:
a fabric enclosure; and
a device for purifying air, the device substantially enclosed by the fabric enclosure, the device for purifying air comprising:
a conduit having an air inlet, an air outlet and a plenum coupling the air inlet to the air outlet;
a sheet at the air inlet, the sheet configured to filter bio aerosols from air entering the conduit,
a light emitting diode (LED) within the conduit, and
a fan at the air outlet.

10. The garment for purifying air as recited in claim 9, further comprising:
a second plenum in the conduit and coupled to the air inlet.

11. The garment of claim 10, wherein at least one of the plenum and the second plenum is a tube.

12. The garment for purifying air as recited in claim 10, further comprising:
a second air outlet at an end region of the second plenum opposing the air inlet; and
a second fan at the second air outlet;
wherein the air outlet and the second air outlet are configured to direct air toward a face of a person wearing the garment.

13. The garment for purifying air as recited in claim 9, further comprising:
a substrate substantially surrounded by the conduit, wherein the substrate includes the LED on at least one surface of the substrate.

14. The garment for purifying air as recited in claim 13, wherein the substrate includes a plurality of LED's on each of opposing surfaces of the substrate.

15. The garment for purifying air as recited in claim 14, wherein the air inlet is configured to provide air flow through the air inlet substantially perpendicular to air flow through an inlet of the plenum.

16. The garment for purifying air as recited in claim 15, further comprising:
a second air inlet configured to provide air flow through the second air inlet substantially perpendicular to air flow through an inlet of the plenum.

17. A method of purifying air comprising:
filtering air entering an inlet of a container through a filtering sheet at the inlet of the container; and
providing a dose of energy to impede a reproduction capability of organisms within the air in the container throughout the container.

18. The method of purifying air as recited in claim 17, further comprising:
controlling a fan at an outlet of the container to pull air through a conduit.

19. The method of purifying air as recited in claim 17, wherein the dose of energy is provided by a plurality of light emitting diodes.

20. The method of claim 19 further comprising:
expelling air into a user's breathing zone from the container by using a fan positioned at an outlet of the container.
